(19) United States
(12) Patent Application Publication
(10) Pub. No.: US 2018/0133068 A1
(43) Pub. Date: May 17, 2018

(54) Attachable Absorbent Fabric Liner for an Underwear

(71) Applicant: Darrel Gene Aten, Arlington, TX (US)

(72) Inventor: Darrel Gene Aten, Arlington, TX (US)

(21) Appl. No.: 15/815,477

(22) Filed: Nov. 16, 2017

Related U.S. Application Data
(60) Provisional application No. 62/423,092, filed on Nov. 16, 2016.

(51) Int. Cl. A61F 13/15; A61F 13/49; A61F 13/513

(52) U.S. Cl. CPC A61F 13/15203; A61F 13/539; A61F 13/513; A61F 13/49

(57) ABSTRACT

An attachable absorbent fabric liner for underwear discretely protects the interior portion of the underwear from stains. The attachable absorbent fabric liner includes an absorbent fabric liner, a fabric adhesion layer, and a releasable liner. The absorbent fabric liner having lay-flat properties prevents waste fluids from meeting the fabric of the underwear. A fabric adhesion layer adheres the absorbent fabric liner onto the seat portion of the underwear. A releasable liner covers the fabric adhesion layer before the fabric adhesion layer is applied to the underwear. The fabric adhesion layer is coated onto a first face of the absorbent fabric liner. The first face is mounted coincident to the seat portion of the underwear causing the waste fluids to fall directly onto the center of the absorbent fabric liner. The releasable liner is attached across the fabric adhesion layer, opposite to the absorbent fabric liner. The releasable liner prevents the acrylic pressure sensitive adhesive (PSA) from evaporating before the absorbent fabric liner is mounted onto the underwear.

The current application claims a priority to the U.S. Provisional Patent application Ser. No. 62/423,092 filed on Nov. 16, 2016.

### FIELD OF THE INVENTION

The present invention generally relates to an attachable absorbent fabric liner for an underwear. More specifically, a thin, lay-flat absorbent fabric liner coated with a fabric adhesion layer attaches to the seat portion of an underwear and prevents waste fluids from penetrating into the fabric of the underwear.

### BACKGROUND OF THE INVENTION

Children must go through toilet training before a full transition to underwear. During this transitionary period, it may be difficult for children to properly clean themselves after using the bathroom. As a result, the child's underwear may show embarrassing stains and marks that can lead to unsanitary conditions and cause the child to have low self-esteem. Products currently in the market that are aimed at resolving this problem have several flaws. The current products are not composed of a material which is comfortable for the child and which the child can easily detect when he or she is using the product. For instance, conventional products are usually fabricated in the form of pads that are bulky and uncomfortable. Children can easily detect when they are wearing the pads and so can outside observers. This can lead to teasing from cohorts of the child, further damaging the child's self-esteem. Additionally, current products also are primarily for absorbing liquids rather than protecting underwear from stains and marks. However, the pads bunch and form clumps that fail to properly absorb waste fluids.

It is therefore an objective of the present invention to provide an absorbent fabric liner proficient at discretely protecting a child's underwear. The present invention includes an absorbent fabric liner composed of 100% cotton fabric which has superior lay-flat properties allowing the present invention to easily be positioned on the inside of underwear. While inside the underwear, the lay-flat properties allow the absorbent fabric layer to conform to the interior contours of the underwear and remain undetected to an outside observer. The present invention also includes a fabric adhesion layer which firmly secures the present invention to the underwear and keeps the present invention flat while secured to the underwear. A release liner keeps the present invention ready to be used at any time without diminishing the effectiveness of the adhesive. The present invention is comfortable for the child and is designed to be undetectable when the child is wearing an underwear with the present invention.

### BRIEF DESCRIPTION OF THE DRAWINGS

FIG. 1 is a bottom perspective of the present invention showing the releasable liner being partially peeled from the fabric adhesion layer and the absorbent fabric liner.

FIG. 2 shows a detail side view of the present invention showing the liner body and the non-stick coating of the releasable liner mounted onto the fabric adhesion layer and the absorbent fabric liner.

FIG. 3 is a dotted line drawing of the present invention positioned inside an underwear which is shown with solid lines.

FIG. 4 is a front view of the present invention illustrating the truncated oval shape of the absorbent fabric liner.

FIG. 5 is a top perspective view of the present invention showing the absorbent fabric liner positioned coextensive to the releasable liner.

### DETAILED DESCRIPTION OF THE INVENTION

All illustrations of the drawings are for the purpose of describing selected versions of the present invention and are not intended to limit the scope of the present invention.

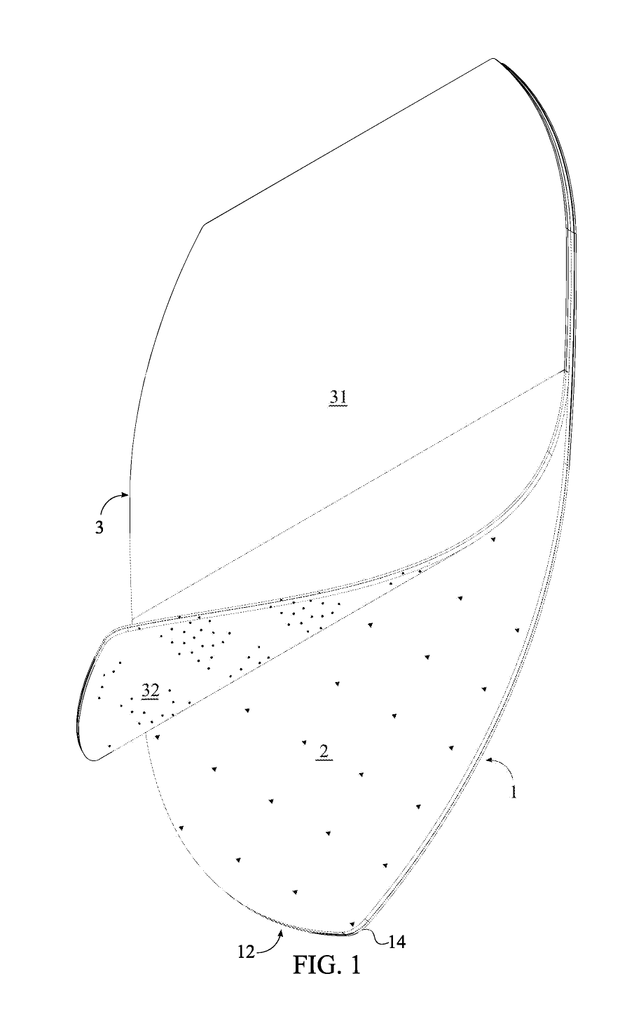

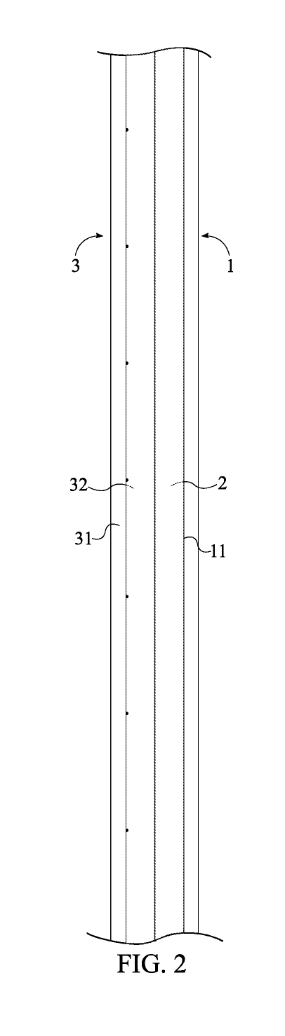

An attachable absorbent fabric liner 1 for underwear worn by children absorbs waste and prevents waste from penetrating the underwear. Referring now to FIG. 1 and FIG. 2, the preferred embodiment of the present invention comprises an absorbent fabric liner 1, a fabric adhesion layer 2, and a releasable liner 3. The fabric adhesion layer 2 enables the absorbent fabric liner 1 to attach to the interior surface of an underwear. The releasable liner 3 prevents the fabric adhesion layer 2 from drying out before the present invention can be used. The fabric adhesion layer 2 is connected across a first face 11 of the absorbent fabric liner 1. The first face 11 of the absorbent fabric liner 1 mounts onto the interior surface of the underwear. Preferably, the first face 11 of the absorbent fabric liner 1 is positioned coincident to the seat of the underwear. The releasable liner 3 is attached across the fabric adhesion layer 2, opposite to the absorbent fabric liner 1. Thus, the releasable liner 3 prevents the fabric adhesion layer 2 from drying out before the absorbent fabric liner 1 can be attached to the underwear. In the preferred embodiment of the present invention, the absorbent fabric liner 1, the fabric adhesion layer 2, and the releasable liner 3 are coextensive to each other. As such, the releasable liner 3 superimposes the fabric adhesion layer 2, ensuring that none of the fabric adhesion layer 2 is exposed. Further, the coextensive configuration of the fabric adhesion layer 2 allows the fabric adhesion layer 2 to completely cover the first face 11 of the absorbent fabric liner 1. This prevents the edges of the absorbent fabric liner 1 from detaching from the interior surface of the underwear.

Referring more particularly to FIG. 2, the preferred releasable liner 3 is designed to easily detach from the fabric adhesion layer 2 once the absorbent fabric liner 1 is ready to be used. As such, the releasable liner 3 comprises a liner body 31 and a non-stick coating 32. The non-stick coating 32 is superimposed across the liner body 31. The non-stick coating 32 allows the absorbent fabric liner 1 to be removed easily from the releasable liner 3 and then applied to the underwear. Similarly, the fabric adhesion layer 2 is attached across the non-stick coating 32, opposite to the liner body 31. The non-stick coating 32 permits the fabric adhesion layer 2 to detach while preserving the adhesive properties of the fabric adhesion layer 2.

The preferred embodiment of the liner body 31 is made of bleached white kraft paper. Moreover, the preferred liner body 31 has a caliper thickness of 6.9 mils (+/−10%) and a weight of 143 grams per meter cubed (g/m³). Alternate embodiments of liner body 31 may be composed of any other material that suits the specific application.

Similarly, the preferred embodiment of the non-stick coating 32 is made of silicone. More specifically, the silicone is coated onto surface of the liner body 31 that mounts coincident onto the first face 11 of the absorbent fabric liner 1. Alternately, the non-stick coating 32 can be made of any other material that removably attaches to the absorbent fabric liner 1.

In the preferred embodiment of the present invention, the releasable liner 3 is approximately 0.17 millimeter (mm) thick. This is the thickness of the liner body 31 and the non-stick coating 32 configured together. Alternate embodiments may have a different thickness based on the type of liner body 31 and non-stick coating 32 utilized.

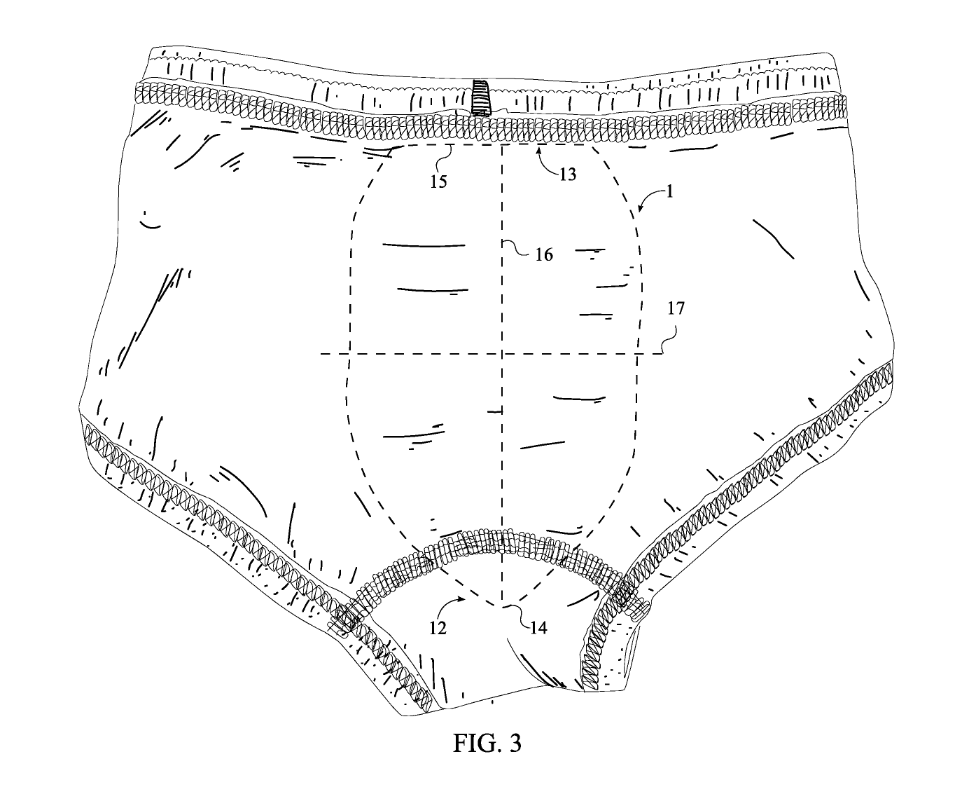

FIG. 3 shows the present invention fitted inside a Boys 2T-3T or Girls 2T-3T underwear. In particular, the present invention is mounted on the seat of the underwear in direct contact with the waste fluids. A profile of the absorbent fabric liner 1 is a truncated oval shape. The truncated oval shape allows the absorbent fabric liner 1 to fit between the leg openings. This ensures that waste fluids spill directly onto the center of the absorbent fabric liner 1. A first end 12 of the truncated oval shape tapers to a blunt tip 14. As such, the first end 12 of the truncated oval shape is coextensive with the crotch panel of the underwear. This prevents waste fluids from leaking out of the bottom portion of the underwear. A second end 13 of the truncated oval shape tapers to a straight edge 15. The second end 13 of the truncated oval shape is positioned coincident to the elastic waistband of the underwear. The blunt tip 14 is positioned coincident to a major axis 16 of the truncated oval shape. Similarly, the straight edge 15 is positioned perpendicular to the major axis 16 of the truncated oval shape. Waste fluids generally spread across a spillage area that extend from the crotch panel to the waistband. Thus, the major axis 16 is longitudinally aligned along the spillage area to prevent the waste fluids from leaking out of the top or bottom edges of the absorbent fabric liner 1. In order to make the present invention as non-intrusive as possible, it is desirable to completely hide the absorbent fabric liner 1 beneath the underwear. In the preferred embodiment of the present invention, the first end 12 of the truncated oval shape tapers at a 45-degree angle up to the blunt tip 14. The first end 12 of the truncated oval shape is thus coextensive to the crotch panel of the underwear, and thus completely hidden from view. Alternately, the taper angle may be any angle needed to hide the first end 12 of the truncated oval shape beneath the crotch panel.

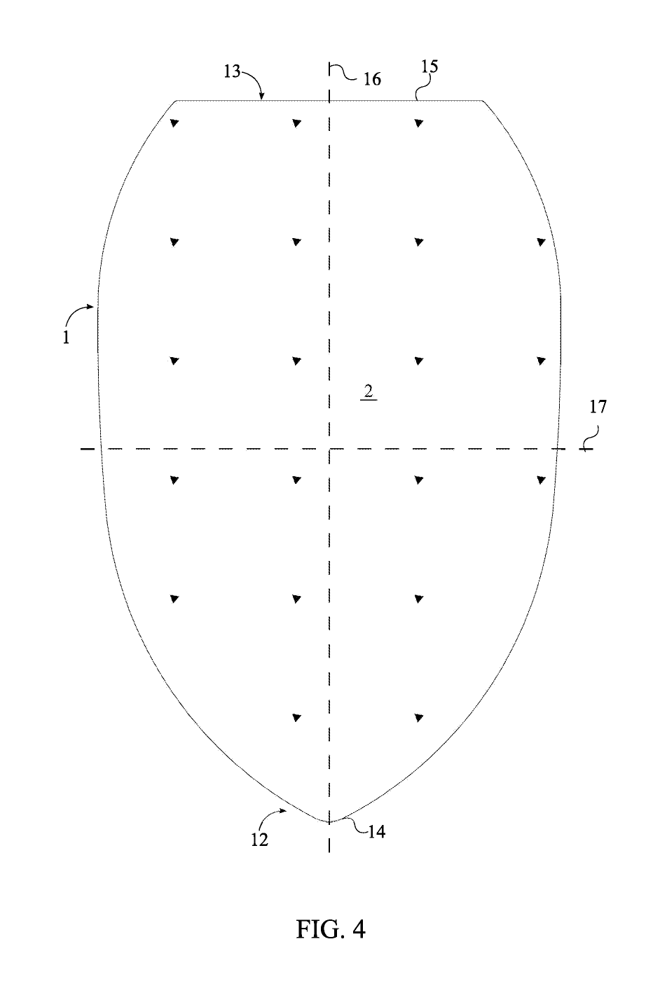

Referring more specifically to FIG. 4, the major axis 16 of the truncated oval shape preferably extends from the crotch panel at the bottom of the underwear to the elastic waistband at the top. The preferred embodiment of the present invention is sized to fit boys 2T-3T and girls 2T-3T underwear. As such, a length of the truncated oval shape along the major axis 16 of the truncated oval shape is approximately 14 centimeters (cm). This maximizes the area of the seat portion covered by the absorbent fabric liner 1, while ensuring that the absorbent fabric liner 1 remains perimetrically enclosed by the underwear. A minor axis 17 positioned perpendicular to the major axis 16 determines the width of the truncated oval shape. In the preferred embodiment of the present invention, the width of the truncated oval shape along the minor axis 17 of the truncated oval shape is 8.9 cm. The width of the absorbent fabric liner 1 is dimensioned to only cover the spillage area while leaving most of the seat portion uncovered. The specific height and the width of the truncated oval shape are thus dimensioned to cover the spillage area while remaining undetectable to the wearer. Alternately, the length of major axis 16 of the truncated oval shape may change in order to suit the underwear having alternate dimensions.

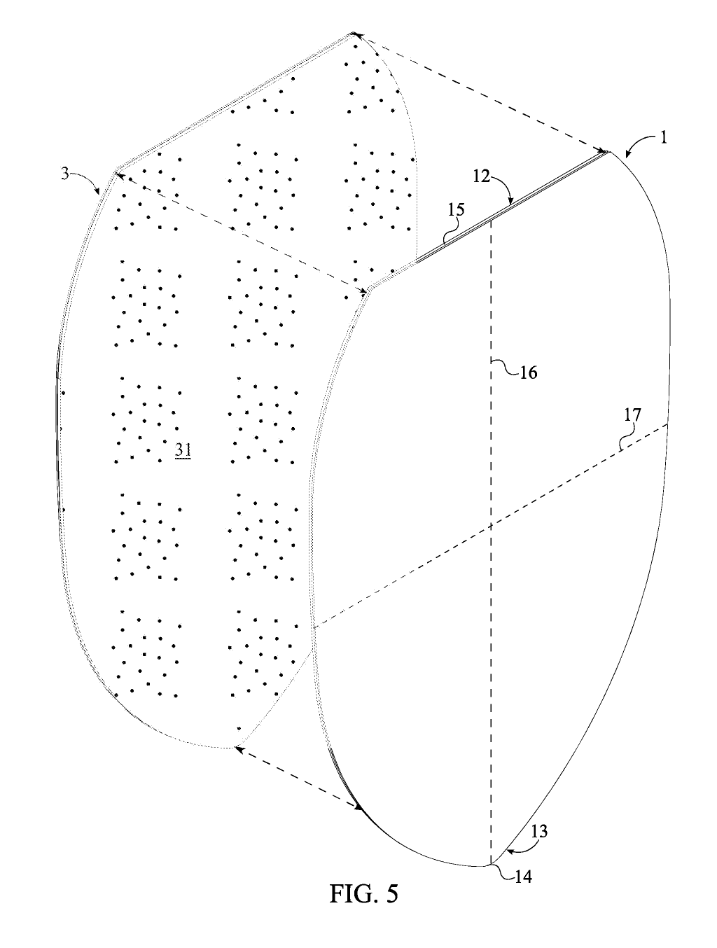

In reference to FIG. 5, to further reduce the chance of detection, the preferred embodiment of the absorbent fabric liner 1 is made of cotton fabric. The cotton fabric is knit using natural cotton filaments, in a low twist style of knit. In the preferred embodiment, the cotton fabric is configured into a jersey knit. Jersey knit has superior lay flat′ characteristics that lies flush against the contours of the underwear. Thus, jersey knit formation substantially reduces the thickness of the present invention, allowing the absorbent fabric liner 1 to lie flat on the seat portion of the underwear. The absorbent fabric liner 1 thus remains completely flush against the seat of the underwear and completely undetectable from the outside. The jersey knit formation also allows the absorbent fabric liner 1 to stretch as required and remain securely attached to the underwear as the underwear bends and flexes according to the movement of the wearer. Using jersey knit cotton fabric allows for an exceptionally thin absorbent fabric liner 1. In the preferred embodiment of the present invention, the absorbent fabric liner 1 is approximately 0.32 millimeters in thickness. Alternately, the thickness may vary according to the materials and techniques used to fabricate the absorbent fabric liner 1.

Referring back to FIG. 2, the jersey knit cotton fabric is preferably manufactured in continuous rolls, 91.44 meters (m) in length and 1.63 m in width. An “edge dressing” is applied to the material to a width of 5.08 cm on each edge which prevents material curl and provides a continuous flat sheet of cotton. The sheet of cotton may preferably have a weight of 82.21 grams (g) to 124.74 g per 30.48 cm of length. Additionally, the cotton may have a minimum number of twist per unit length of filament. The term twist refers to the number of turns per unit of length of a single cotton filament. This may be expressed as turns per inch (tpi), turns per meter (tpm), or turns per centimeter (tpcm). The twist per length is designated as low, moderate, or high. Once the cotton sheet is unwound from the roll, the absorbent fabric liner 1 is created by cutting the sheet of cotton into the truncated oval shape.

Together, the absorbent fabric liner, the fabric adhesion layer, and the releasable liner are configured to approximately weigh 3.5 grams. This is a consequence of the specific materials utilized to make these components and may change in alternate embodiments, wherein the different materials utilized.

The preferred fabric adhesion layer 2 is made of an acrylic pressure-sensitive adhesive (PSA). Additionally, the acrylic PSA is preferably FDA approved for human contact. In addition to providing a means to attach the absorbent fabric liner 1 to the underwear, the acrylic PSA also provides a moderate moisture barrier so that waste fluids do not penetrate through to the underwear. In alternate embodiments, any fabric adhesion layer 2 may utilize any adhesive material.

Although the invention has been explained in relation to its preferred embodiment, it is to be understood that many other possible modifications and variations can be made without departing from the spirit and scope of the invention as hereinafter claimed.

What is claimed is:

1. An attachable absorbent fabric liner for undergarment comprises:
an absorbent fabric liner;
a fabric adhesion layer;
a releasable liner;
the fabric adhesion layer being connected across a first face of the absorbent fabric liner;
the releasable liner being attached across the fabric adhesion layer, opposite to the absorbent fabric liner; and
the absorbent fabric liner, the fabric adhesion layer, and the releasable liner being coextensive to each other.

2. The attachable absorbent fabric liner for undergarment as claimed in claim 1 comprises:
the release liner comprises a liner body and a non-stick coating;
the non-stick coating being superimposed across the liner body; and
the fabric adhesion layer being attached across the non-stick coating, opposite to the liner body.

3. The attachable absorbent fabric liner for undergarment as claimed in claim 2, wherein the liner body is made of bleached white kraft paper.

4. The attachable absorbent fabric liner for undergarment as claimed in claim 2, wherein the non-stick coating is made of silicone.

5. The attachable absorbent fabric liner for undergarment as claimed in claim 1 comprises:
a profile of the absorbent fabric liner being a truncated oval shape;
a first end of the truncated oval shape tapering to a blunt tip;
a second end of the truncated oval shape tapering to a straight edge;
the blunt tip being positioned coincident to a major axis of the truncated oval shape; and
the straight edge being positioned perpendicular to the major axis of the truncated oval shape.

6. The attachable absorbent fabric liner for undergarment as claimed in claim 5, wherein the first end of the truncated oval shape tapers at a 45-degree angle up to the blunt tip.

7. The attachable absorbent fabric liner for undergarment as claimed in claim 5, wherein a length of the truncated oval shape along the major axis of the truncated oval shape is approximately 14 centimeters (cm).

8. The attachable absorbent fabric liner for undergarment as claimed in claim 5, wherein a width of the truncated oval shape along a minor axis of the truncated oval shape is approximately 8.9 cm.

9. The attachable absorbent fabric liner for undergarment as claimed in claim 1, wherein the absorbent fabric liner is made of cotton fabric.

10. The attachable absorbent fabric liner for undergarment as claimed in claim 9, wherein the cotton fabric is configured into a jersey knit.

11. The attachable absorbent fabric liner for undergarment as claimed in claim 1, wherein the fabric adhesion layer is made of an acrylic pressure-sensitive adhesive.

12. The attachable absorbent fabric liner for undergarment as claimed in claim 1, wherein a thickness of the absorbent fabric liner is approximately 0.32 millimeters (mm).

13. The attachable absorbent fabric liner for undergarment as claimed in claim 1, wherein a thickness of the releasable liner is approximately 0.17 mm.

14. The attachable absorbent fabric liner for undergarment as claimed in claim 1, wherein the absorbent fabric liner, the fabric adhesion layer, and the releasable liner are configured to approximately weigh 3.5 grams.

15. An attachable absorbent fabric liner for undergarment comprises:
an absorbent fabric liner;
a fabric adhesion layer;
a releasable liner;
the fabric adhesion layer being connected across a first face of the absorbent fabric liner;
the releasable liner being attached across the fabric adhesion layer, opposite to the absorbent fabric liner;
the absorbent fabric liner, the fabric adhesion layer, and the releasable liner being coextensive to each other;
the release liner comprises a liner body and a non-stick coating;
the non-stick coating being superimposed across the liner body;
the fabric adhesion layer being attached across the non-stick coating, opposite to the liner body;
a profile of the absorbent fabric liner being a truncated oval shape;
a first end of the truncated oval shape tapering to a blunt tip;
a second end of the truncated oval shape tapering to a straight edge;
the blunt tip being positioned coincident to a major axis of the truncated oval shape; and
the straight edge being positioned perpendicular to the major axis of the truncated oval shape.

16. The attachable absorbent fabric liner for undergarment as claimed in claim 15, wherein the first end of the truncated oval shape tapers at a 45-degree angle up to the blunt tip, wherein a length of the truncated oval shape along the major axis of the truncated oval shape is approximately 14 centimeters (cm), and wherein a width of the truncated oval shape along a minor axis of the truncated oval shape is approximately 8.9 cm.

17. The attachable absorbent fabric liner for undergarment as claimed in claim 15, wherein the absorbent fabric liner is made of cotton fabric.

18. The attachable absorbent fabric liner for undergarment as claimed in claim 17, wherein the cotton fabric is configured into a jersey knit.

19. The attachable absorbent fabric liner for undergarment as claimed in claim 15, wherein the fabric adhesion layer is made of an acrylic pressure-sensitive adhesive.

20. The attachable absorbent fabric liner for undergarment as claimed in claim 1, wherein a thickness of the absorbent fabric liner is approximately 0.32 millimeters (mm), wherein a thickness of the releasable liner is approximately 0.17 mm, and wherein the absorbent fabric liner is configured to approximately weigh 3.5 grams.
